(19) United States
(12) Patent Application Publication
(10) Pub. No.: US 2016/0228048 A1
(43) Pub. Date: Aug. 11, 2016

(54) METHOD OF QUANTIFYING MELANIN MASS DENSITY IN VIVO

(71) Applicant: NATIONAL TAIWAN UNIVERSITY, Taipei (TW)

(72) Inventors: CHI-KUANG SUN, Taipei (TW); WEI-MIN LIU, Taipei (TW)

(21) Appl. No.: 14/614,532

(22) Filed: Feb. 5, 2015

(51) Int. Cl. A61B 5/00; A61B 5/1455

(52) U.S. Cl. CPC A61B 5/443; A61B 5/1455; A61B 5/742; A61B 2576/02

(57) ABSTRACT

A method of quantifying melanin includes receiving at a data processor optical data produced by an optical sensor which receives a light from a tissue; determining a set of intensity values based on the optical data; for each intensity value, converting the intensity value to a quantifying value corresponding to quantity of melanin in the tissue based on a two-dimensional non-linear regression with one variable representing the intensity value and another variable representing the quantity of melanin; collecting at the data processor each quantifying value; generating at the data processor melanin quantity distribution data according to the quantifying values; and outputting the melanin quantity distribution data to be presented on a medium.

### FIELD

The subject matter herein generally relates to a method of quantifying melanin mass density in vivo.

### BACKGROUND

People are often afflicted with skin pigment disorders visible as dark or white patches on the skin. The dark or white patches on the skin are often due to abnormal variation in the quantity of melanin in the skin. Methods of quantifying melanin mass density in the skin are of interest in the study of skin pigment disorders and in determining whether a skin pigment disorder is due to a malignancy that would require medical treatment.

In particular, it is useful to analyze the quantity of melanin in the skin in order to study the genetics and regulation of melanogenesis in melanocytes. Melanin quantity estimation is useful for diagnosis, preoperative assessment, and therapeutic monitoring. For the purpose of diagnosis, melanin may play as a natural biomarker of melanocyte activity and is a promising marker to differentiate benign, dysplastic, and malignant tissue.

There are several melanin quantity estimation methods including High Performance Liquid Chromatography (HPLC), Electron Paramagnetic Resonance (EPR), Absorption Spectroscopy (AS), Diffuse Reflectance Spectroscopy (DRS), Raman Spectroscopy (RS), Two-Photon Excited Fluorescence Microscopy (TPEFM), Reflectance Confocal Microscopy (RCM), Photoacoustic Microscopy (PAM), and Pump-Probe Microscopy (PPM). Out of the methods listed above, HPLC is the most widely accepted one nowadays.

HPLC is based on the chemical degradation of the melanin polymer and HPLC analysis of the specific degradation products. The eumelanin polymer is degraded into pyrrole-2,3,5-tricarboxylic acid (PTCA) by KMnO₄ oxidation, while pheomelanin is split into aminohydroxyphenylalanine (AHP) isomers by reductive hydrolysis with hydroiodic acid (HI). The formula of the degradation process is still under testing to make the evaluation easier to perform and to extract more accurate information from the pigmented specimen.

### BRIEF DESCRIPTION OF THE DRAWINGS

Implementations of the present technology will now be described, by way of example only, with reference to the attached figures.

FIG. 1 is a schematic view of a procedure of quantifying melanin mass density in vivo.

FIG. 2 is a schematic view of a process of finding intensity value of background third harmonic generation (THG) and using the intensity value of background THG when converting optical data to obtain a THG ratio for each pixel.

FIG. 3 is a block diagram illustrating a system for quantifying melanin mass density in vivo.

FIG. 4 is a block diagram illustrating a processor module and memory module of an in vivo quantification of melanin mass density system.

FIG. 5 shows images of melanin mass density of different Fitzpatrick skin types.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

The present disclosure is described in relation to a method of quantifying melanin mass density, especially in vivo. In general, melanin is quantified by obtaining optical data produced by an optical sensor which receives a light from a tissue, obtaining intensity value by processing the optical data, and converting the intensity value of the optical data to a quantifying value corresponding to quantity of melanin in the tissue based on a two-dimensional non-linear regression with one variable representing the intensity value of the optical data and another variable representing the quantity of melanin. For example, the optical data can be obtained in such a way that the intensity value is a function of the melanin mass density in the tissue, and the two-dimensional non-linear regression may have been performed on empirical data obtained by scanning melanin samples of known mass densities. As further described below, optical data indicative of melanin mass density may be obtained by scanning tissue with light source at an excitation wavelength of about 1230 nm, and receiving a light from the tissue at a third harmonic of the excitation wavelength (i.e. about 410 nm). The quantification of melanin may also include a background correction applicable to the kind of tissue being scanned.

In one embodiment, skin is scanned by a microscope in vivo. An excitation light with 1230 nm wavelength is guided through the microscope and directed to the skin surface of an animal. A photo sensor is configured to obtain a non-linear light such as third harmonic (410 nm) or two harmonic (615 nm) generation reflected from the skin tissue. After the non-linear light is detected, the optical signals are transformed into corresponding digital signals and then stored in a storage medium.

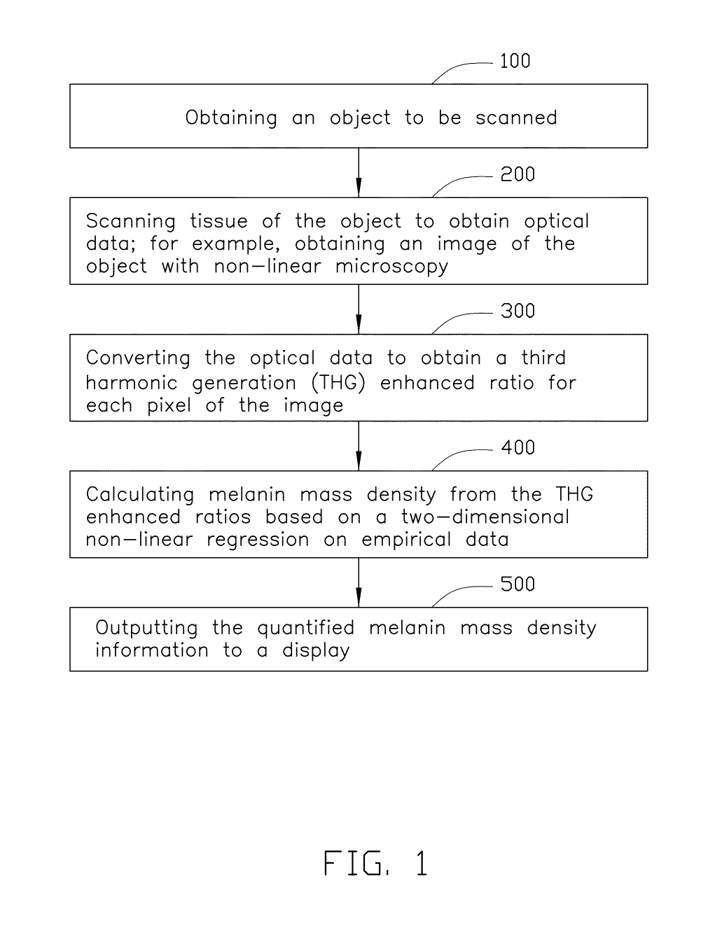

FIG. 1 illustrates an example of a specific procedure of quantifying melanin mass density in vivo. The quantification of melanin mass density can be achieved by the following operations:
in box 100, obtaining an object to be scanned;
in box 200, scanning tissue of the object to obtain optical data; for example, obtaining an image of the object with non-linear microscopy;
in box 300, converting the optical data to obtain a third harmonic generation (THG) enhanced ratio for each pixel of the image;
in box 400, calculating melanin mass density from the THG enhanced ratios based on a two-dimensional non-linear regression on empirical data; and
in box 500, outputting the quantified melanin mass density information to a display.

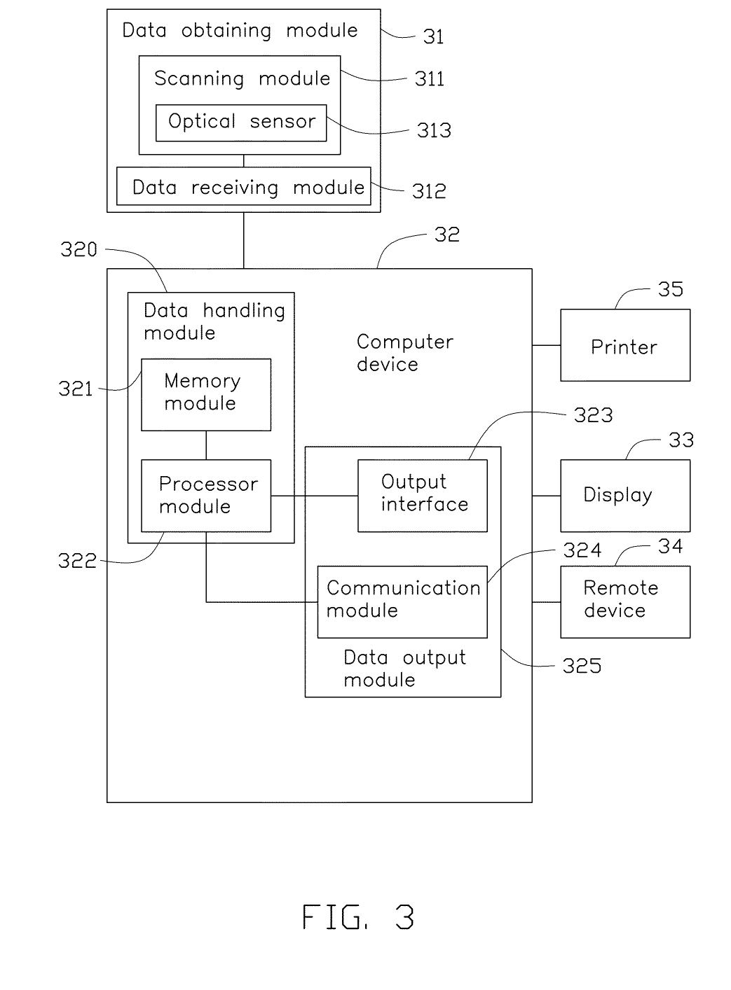

A THG image may be obtained in box 200. An example of a procedure of obtaining a THG image may be found in FIG. 3 of the Taiwan patent application TW099139177, published May 16, 2012 under Publication No. TW201219774A1, incorporated herein by reference.

In this example, the non-linear microscopy may comprise a mode-locked chromium-forsterite laser with center wavelength 1230 nm, which may apply a light source. The excitation beam is collimated and guided into a scanning system connected with a modified inverted microscope for in vivo skin imaging. A pair of galvanometer mirrors in the scanning system may provide the 2-D scanning of the laser beam. The scanning beam may be focused onto the human skin. The endogenous THG signal may be then collected from epidermis and reflected by a dichroic beam splitter. THG signal may be separated by another dichroic beam splitter and sent to a photomultiplier (PMT) with bandpass filters inserted in the optical path of the PMT. Multiple imaging modes can use a beam splitter and a respective bandpass filter and photomultiplier tube (PMT) for each imaging modality.

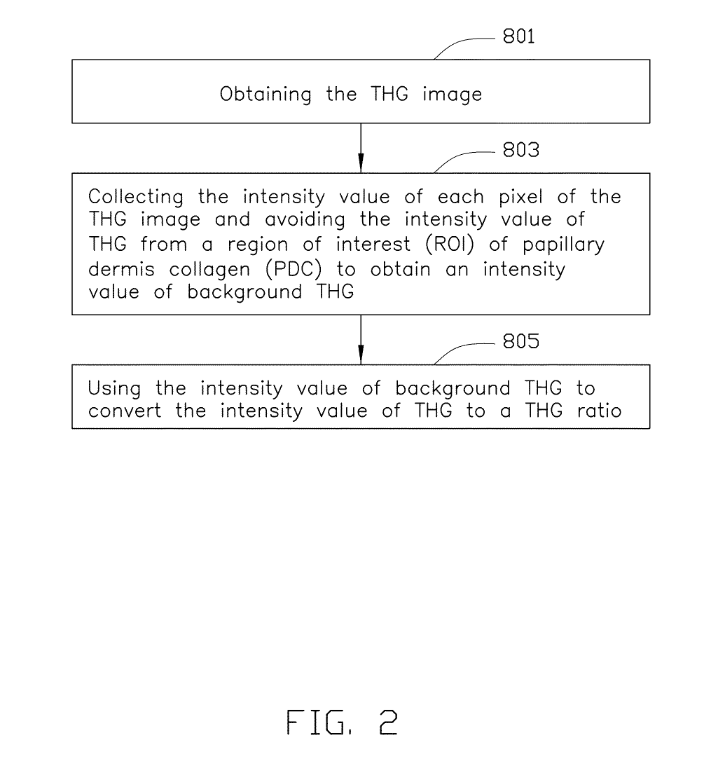

FIG. 2 illustrates the process of finding the intensity value of background THG. The intensity value of background THG can be found by the following operations:
in box 801, obtaining the THG image;
in box 803, collecting the intensity value of each pixel of the THG image and avoiding the intensity value of THG from a region of interest (ROI) of papillary dermis collagen (PDC) to obtain an intensity value of background THG;
in box 805, using the intensity value of background THG to convert the intensity value of THG to a THG ratio.

In box 801, the THG image is obtained from the previous box 200 of FIG. 1. Then, in box 803, the information including position and intensity value of a background THG is avoided from the THG image. Once the background THG is obtained, in box 805, a THG enhanced ratio is converted by avoiding the background THG from each pixel of the THG image through a convertor. The convertor can be a computer device.

A background THG may be defined as THG not reflected from melanin where such THG might be generated from large sized membrane organelles, including Golgi apparatus, endoplasmic reticulum (ER), and mitochondria or cytoplasmic ground substance.

In box 803, one could select a region of interest of papillary dermis collagen, where no melanin exists, to collect an intensity value of THG (an implication of which the intensity value of THG collected there is unwanted).

Also, the selection of ROI of PDC may avoid certain regions with background intensity value of THG. For example, the selection of ROI of PDC may avoid regions including: capillaries, regions with vague morphology of collagen or basal cells, and other unknown regions that may or may not have THG intensity value. The THG intensity value found in said regions represents background intensity value.

Since study shows the background (without melanin) THG intensity value is very similar (with a ratio of 0.99) to that of PDC, one could divide the intensity value of THG from the ROI of PDC by 0.99 to get an intensity value of background THG. Therefore, in box 805, the converter may divide the intensity value of THG from each pixel by the intensity value of background THG in order to obtain a THG enhanced ratio for each pixel of the THG image. Thus, in box 805, the optical data may be corrected with the background value to remove the background data from the optical data.

In another embodiment of the present invention, a collection of data involving albino skin is used. (steps not shown in the figures) An albino THG ratio is obtained by dividing the THG intensity value of each pixel of a sectioned image (obtained from the epidermis layer where melanin usually locates but was absent in albino cells) by the THG background intensity value of each pixel with the corresponding position of a sectioned image (obtained from collagen region in the dermis layer since collagen is known to result in second harmonic generation but only limited THG). An average albino THG ratio can be obtained by averaging all albino THG ratio of all pixel data obtained in one albino. Further, more than one albino skin may be used to calculate an inter-personal averaged albino THG ratio if desirable. The albino THG ratio, either the ratio of single pixel data point, the average ratio of plural pixel data points, or the average ratio of multiple albino data, is used as a background-calibrating standard.

The THG ratio of a non-albino participant is obtained by the same approached as described in the albino protocol. The difference is that melanin locates and exists in the cells in the epidermis layer in normal participant but not in albino cells. Such difference usually leads to a greater THG intensity value obtained from the epidermis layer than the value in an albino. By dividing the THG ratio of a non-albino normal participant to the albino THG ratio, a THG enhancement ratio (a melanin enhancement ratio) is obtained. Such THG enhancement ratio explains, in human or animal skin, the difference of THG intensity enhanced by the existence of melanin and is a calibrated or corrected value for later melanin mass density calculation. Most following embodiments and steps are illustrated by using the THG enhanced ratio but one would understand such THG enhanced ratio may also be substituted by the THG enhancement ratio by employing albino data.

An example method for calculating melanin mass density in box 400 of FIG. 1 contains two equations. The equations may be based on two-dimensional non-linear regression on a set of data collected from a series of experiments involving two-photon excited fluorescence (TPEF) and a strong linear relation to melanin mass density and position correlation with THG.

The first equation applies when the THG enhanced ratio is less than 5.93 or the melanin mass density (MMD) is less than or equal to 11.0 mg/ml. The first equation is:

THG enhanced ratio=1.19*10⁻³MMD^{3.47}+1.06

The second equation applies when the THG enhanced ratio is greater than 5.93 or the melanin mass density is greater than 11.0 mg/ml. The second equation is:

THG enhanced ratio=5.04*10⁻¹MMD^{0.95}+1.06

For example, the non-linear microscopy apparatus used for collecting the THG data is calibrated by collecting optical data from melanin standards. Suitable melanin standards can be prepared by dispersing different selected amounts of melanin in quantities of solvent. For example, synthetic melanin prepared by the chemical oxidation of tyrosine (Sigma Aldrich Chemical Co., Cat. No. M8631) is dispersed in a 1M NaOH solvent to make a number of melanin standard solutions in the range of about 0 to 5 mg/ml. The melanin can be dispersed by shaking the melanin in the NaOH solvent for ten seconds followed by sonicating for 30 minutes in a water bath at 40° C. The THG intensities from the standard solutions, when imaged in the non-linear microscopy apparatus, are then correlated with the known melanin concentrations by applying two-dimensional non-linear regression.

A simple example of two-dimensional non-linear regression analysis may be plotting the THG intensity value of the data points on one axis (x) of a sheet of log-log paper, and plotting the MMD of the data points on the other axis (y) of the sheet of log-log paper, and then fitting a line through the data points. In practice, the line can be fitted by a computer program that applies the well-known technique of “least squares” to minimize the sum of the distances from the line to the data points. Also, in practice it has been found that the correlation between THG intensity value and MMD can be more precisely represented by fitting two line segments to the data points. The two line segments result in the two equations above for calculating melanin mass density in box 400 of FIG. 1.

The microscopy set-up described above can be used for two-photon-excited fluorescence (TPEF) as well as THG. For TPEF, the optical detector detects an optical signal at about twice the excitation frequency, so an optical bandpass filter at about twice the excitation frequency may be placed in the optical path to the detector. By using a beam splitter to split the optical path into a first path to the TPEF detector and a second path to the THG detector, it may be possible to record a TPEF signal and a THG signal simultaneously.

In contrast to THG, TPEF provides a signal that is more linear in proportion to MMD. THG, however, is more suitable for measuring the melanin content of human skin in vivo because TPEF has a reduced penetration depth in strongly pigmented tissue. Due to these relative advantages and disadvantages, TPEF can be used for calibration of the THG, and then the THG can be used for human skin in vivo measurements of MMD.

TPEF provides a way of identifying regions free of melanin and therefore suitable ROI for THG background measurement. For example, in the microscopy set-up described above, calibration of the TPEF with synthetic melanin standard solutions results in a set of data points having a highly linear relationship between TPEF intensity value and MMD. Linear regression may be used to correlate the TPEF with the quantified synthetic melanin resulting in an equation such as:

TPEF=68.01×MMD+390.8

Consequently, pixels in the TPEF image of a tissue sample from this apparatus having a TPEF value less than 390.8 are assumed to be from locations without melanin. Thus, the TPEF can be used for locating ROI for THG background.

A spectrophotometer may be used for the optical detector for TPEF. The TPEF spectra can distinguish between eumelanin and pheomelanin. The TPEF spectrum of eumelanin peaks at 615-625 nm, and the TPEF spectrum of pheomelanin peaks at 640-680 nm. Therefore, it is possible to use TPEF to straightforwardly measure the relative concentrations of eumelanin and pheomelanin in a tissue sample, and to better calibrate these measurements against the TPEF signal from quantified synthetic melanin.

FIG. 3 is a block diagram illustrating a system for quantifying melanin mass density in vivo. This system for quantifying melanin comprises a data obtaining module 31, a computer device 32, a display 33, and a remote device 34. The data obtaining module 31 comprises a scanning module 311 and a data receiving module 312 coupled with the scanning module 311. The computer device 32 comprises a data handling module 320 and a data output module 325. The data handling module 320 comprises a memory module 321 and a processor module 322. The data output module 325 comprises an output interface 323 and a communication module 324.

In this embodiment, the scanning module 311 may be used to obtain an in vivo skin image from an observed object. In one embodiment, a non-linear optical microscopy can be used. The scanning module 311 comprises an optical sensor 313. The scanning module 311 is configured to apply a light source to a tissue. The in vivo skin optical data is produced by an optical sensor 313 which receives a light from a tissue. The optical data may be collected from the data receiving module 312 and sent to the processor module 322 and the memory module 321 of the computer device 32. The processor module 322 executes a software program to determine ROI and calculate THG enhanced ratio and melanin mass density. (Alternatively (steps not shown in the figures), THG enhancement ratio, determined by calibrating albino THG ratio as described previously, can be used here and generate melanin mass density value.) The image processed by the processor module 322 and the memory module 321 can output through the output interface 323, and be presented on the display 33 or printed on an printer 35. The processed image also can be stored or transferred to the remote device 34 by the communication module 324.

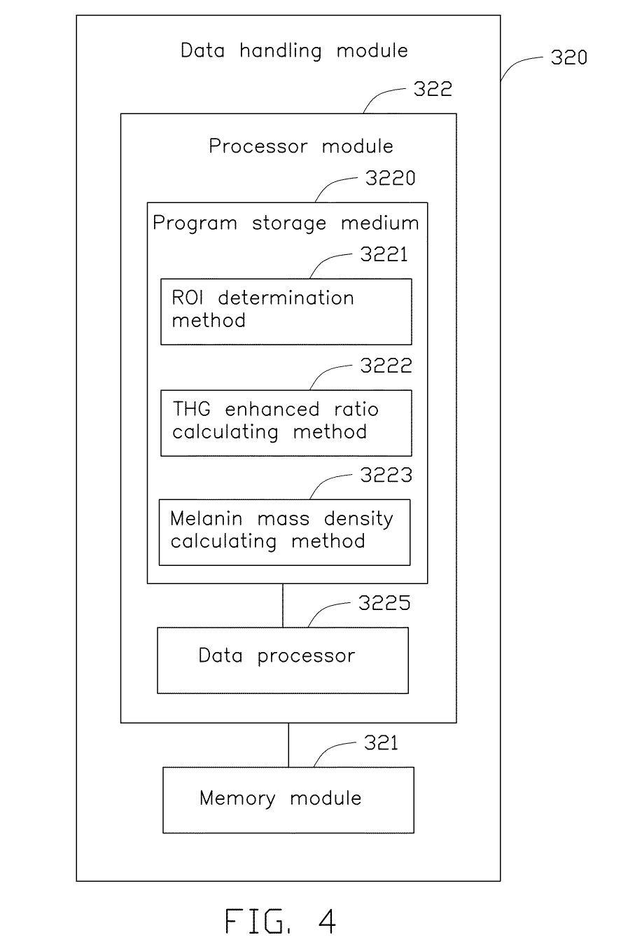

FIG. 4 is a block diagram illustrating the data handling module 320 of the system for quantifying melanin mass density in vivo. The data handling module 320 comprises a processor module 322 and a memory module 321. The processor module 322 further comprises a non-transitory program storage medium 3320 and a data processor 3225. For example, the program storage medium 3320 may be a disk storage or a flash memory. The program storage medium 3320 contains software routines of computer program instructions executable by the data processor 3225. These software routines include an ROI determination method 3221, a THG enhanced ratio calculating method 3222 and a melanin mass density calculating method 3223.

The data processor 3225 executes methods of the ROI determination method 3221, the THG enhanced ratio calculating method 3222 and the melanin mass density calculating method 3223, while the memory module 321 stores the result after data is calculated. The ROI determination method 3221 may be executed to select ROI of PDC of an in vivo skin image. The THG enhanced ratio calculating method 3222 may be executed to calculate the ratio by dividing the intensity value of THG from each pixel by the intensity value of background THG. (Alternatively (steps not shown in the figures), the THG enhanced ratio is substituted by the THG enhancement ratio, which is calculated by dividing the THG ratio of a normal skin to the albino THG ratio as previously described.) The melanin mass density calculating method 3223 may be executed to calculate the melanin mass density with the result from the THG enhanced ratio calculating method 3222.

For example, the melanin mass density calculating method calculates MMD from the THG enhanced ratio by the inversion of the above two equations, according to:

If THG enhanced ratio<5.93, then MMD=((THG_{enhanced ratio}−1.06)/1.19×10⁻³)^{(1/3,47)}

If THG enhanced ratio>5.93, then MMD=((THG_{enhanced ratio}−1.06)/5.04)^{(1/0.95)}

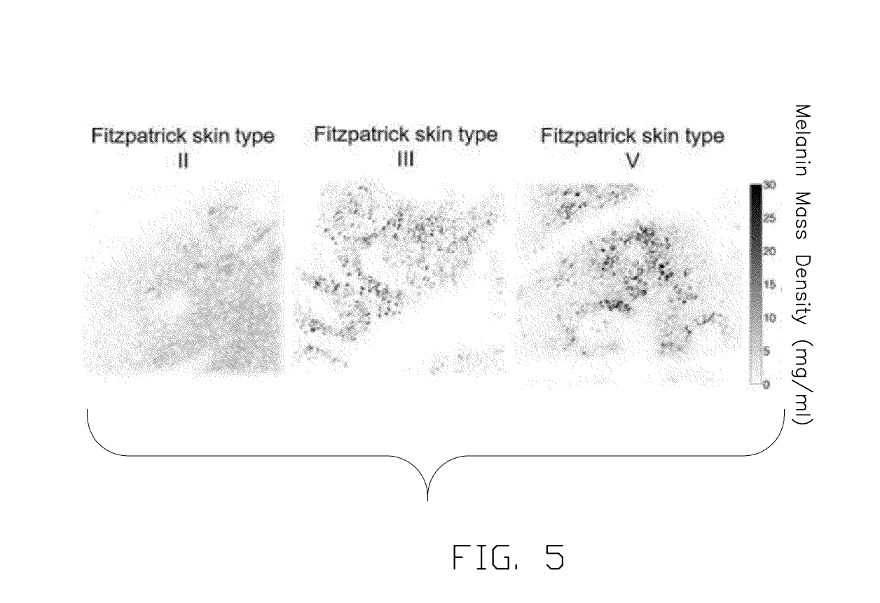

FIG. 5 illustrates three images of melanin mass density of different Fitzpatrick skin types. In particular, FIG. 5 shows a first image of a Fitzpatrick skin type II, a second image of a Fitzpatrick skin type III, and a third image of a Fitzpatrick skin type IV. FIG. 5 also shows a gray-scale bar labeled with a scale correlating the black-and-white intensity value of each pixel in the images of melanin mass density to a numeric value of MMD. An entirely white pixel has a MMD of zero, and an entirely black pixel has a MMD of at least thirty mg/ml.

The embodiments illustrate and described above are only examples. Many details are often found in the art of non-linear microscopy and two-photon excited fluorescence. Therefore, many such details are neither illustrated nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It is also understandable that the present invention is to all types of skins including human beings and other animals or plants. It will therefore be appreciated that the embodiments described above may be modified within the scope of the claims.

What is claimed is:

1. A method of quantifying melanin comprising:
(a) receiving at a data processor optical data produced by an optical sensor which receives a light from a tissue;
(b) determining a set of intensity values based on the optical data;
(c) for each intensity value, converting the intensity value to a quantifying value corresponding to quantity of melanin in the tissue based on a two-dimensional non-linear regression with one variable representing the intensity value and another variable representing the quantity of melanin;
(d) collecting at the data processor each quantifying value;
(e) generating at the data processor melanin quantity distribution data according to the quantifying values; and
(f) outputting the melanin quantity distribution data to be presented on a medium.

2. The method of claim 1, wherein the optical data is obtained in vivo.

3. The method of claim 1, which further comprises presenting the optical data as an image.

4. The method of claim 3, wherein the converting the intensity value to a quantifying value corresponding to quantity of melanin in the tissue is applied to every pixel of the image.

5. The method of claim 1, wherein the tissue is skin.

6. The method of claim 1, wherein the light is a third harmonic generation reflected from the tissue.

7. The method of claim 1, wherein the converting the intensity value to the quantitfying value corresponding to quantity of melanin in the tissue comprises a determination of a background intensity value, wherein the background intensity value is the intensity value of a region of the tissue containing no melanin, and a correction of the intensity value with the background intensity value.

8. The method of claim 7, wherein the correction comprises a removal of the background intensity value from the intensity value of the optical data.

9. The method of claim 8, wherein the background intensity value is an intensity value obtained from albino skin or obtained from a region of skin with substantially no melanin.

10. The method of claim 1, wherein the quantity of melanin in the tissue is a group of numerical data.

11. The method of claim 1, wherein the two-dimensional non-linear regression is derived from a correlation between intensity value of third harmonic generation and amount of quantified synthetic melanin.

12. A system for quantifying melanin comprising:
a data obtaining module configured to receive optical data produced by an optical sensor which receives a light from a tissue;
a data handling module coupled with the data obtaining module and configured to convert an intensity value based on the optical data to a quantifying value corresponding to quantity of melanin in the tissue based on a two-dimensional non-linear regression with one variable representing the intensity value of the optical data and another variable representing the quantity of melanin in the tissue; and
an output module coupled with the data handling module and configured to output the quantity of melanin in the tissue.

13. The system of claim 12, wherein the data obtaining module is configured to obtain the optical data from a microscope or scanner.

14. The system of claim 12, wherein the data obtaining module is a scanning module configured to obtain the optical data by scanning the tissue.

15. The system of claim 12, wherein the data handling module comprises a memory and a data processor programmed to perform the converting the intensity value based on the optical data to a quantifying value corresponding to quantity of melanin in the tissue.

16. The system of claim 12, wherein the output module is a display for displaying the quantity of melanin in the tissue.

17. The method of claim 12, wherein the quantity of melanin in the tissue is a group of numerical data.

18. The system of claim 17, wherein the output module is a printer for printing the group of numerical data.

19. The system of claim 12, wherein the output module is a communication module for delivering the converted result to a remote device.

20. An apparatus for quantifying melanin, the apparatus comprising:
a scanning module configured to apply a light source to a tissue, wherein an optical data is produced by an optical sensor which receives a light from a tissue;
a data receiving module configured to obtain said optical data;
a data processor coupled to the data receiving module to receive the optical data from the data receiving module;
a non-transitory storage medium coupled to the data processor and storing computer instructions, wherein the computer instructions, when executed by the data processor, convert intensity value of the optical data to quantity of melanin based on a two-dimensional non-linear regression with one variable representing the intensity value of the optical data and another variable representing the quantity of melanin, to produce converted data; and
an output module coupled to the data processor to deliver the converted data.
